(19) United States
(12) Patent Application Publication
(10) Pub. No.: US 2023/0022721 A1
(43) Pub. Date: Jan. 26, 2023

(54) COMPOSITIONS AND METHODS FOR INHIBITION AND INTERRUPTION OF BIOFILM FORMATION

(71) Applicant: DENTSPLY SIRONA Inc., York, PA (US)

(72) Inventors: Xiaoming JIN, Middletown, DE (US); Hui LU, Magnolia, DE (US); Christopher DAMIEN, Marietta, PA (US); Bernard KOLTISKO, Milton, DE (US)

(73) Assignee: DENTSPLY SIRONA Inc., York, PA (US)

(21) Appl. No.: 17/941,323

(22) Filed: Sep. 9, 2022

Related U.S. Application Data
(63) Continuation of application No. 15/476,005, filed on Mar. 31, 2017, now abandoned.
(60) Provisional application No. 62/317,172, filed on Apr. 1, 2016.

(51) Int. Cl. A01N 57/04; A61K 8/81; A61Q 11/00; A01N 33/12; A01N 57/34; A61K 8/55; A61K 8/898; A61K 8/41; A61K 6/20; A61K 6/887; A61B 42/10; A61B 46/00; A23L 33/16; A61C 5/70; A61C 5/50; A01N 25/08; A01N 25/10; A01N 43/50; A23L 2/52; A61B 17/06; A61B 17/84; A61B 17/86; A61C 7/08; A61C 7/16; A61C 8/00; A61C 9/00; A61C 13/00; A61C 19/06; A61F 2/00; A61F 2/06; A61F 2/16; A61F 2/24; A61F 6/04; A61F 6/08; A61F 6/14; A61F 13/20; A61F 13/36; A61M 5/14; A61M 16/00; A61M 25/00; A61M 27/00; F16L 58/04

(52) U.S. Cl. CPC A01N 57/04; A61K 8/817; A61Q 11/00; A61K 8/8158; A01N 33/12; A01N 57/34; A61K 8/55; A61K 8/8188; A61K 8/898; A61K 8/416; A61K 6/20; A61K 6/887; A61B 42/10; A61B 46/40; A23L 33/16; A61C 5/70; A61C 5/50; A01N 25/08; A01N 25/10; A01N 43/50; A23L 2/52; A61B 17/06166; A61B 17/848; A61B 17/86; A61C 7/08; A61C 7/16; A61C 8/0013; A61C 8/0048; A61C 9/00; A61C 13/0004; A61C 13/0022; A61C 19/066; A61F 2/0063; A61F 2/06; A61F 2/16; A61F 2/24; A61F 6/04; A61F 6/08; A61F 6/14; A61F 13/2051; A61F 13/36; A61M 5/14; A61M 16/0057; A61M 25/0043; A61M 27/002; F16L 58/04; A23V 2002/00; A61B 2017/00889

(57) ABSTRACT

Compositions and methods for inhibiting and interrupting biofilm formation, and for destabilizing established biofilms are provided, the novel compositions including polymeric resins and monomeric non-polymerizable and polymerizable resins. More particularly, the compositions and methods enable the protection and removal of biofilms from surfaces in the context of medical, consumer, domestic, food service, environmental and industrial applications, where the effects constitute beneficial and desirable biofilm attenuating activity.

This application is a continuation application of pending U.S. patent application Ser. No. 15/476,005 filed Mar. 31, 2017.

### FIELD OF THE INVENTION

The disclosure relates to compositions and methods for inhibiting and interrupting biofilm formation, and for destabilizing established biofilms. More particularly, the disclosure provides compositions and methods that enable the protection and removal of biofilms from surfaces in the context of medical, consumer, domestic, food service, environmental and industrial applications. In accordance with the various embodiments, the effects constitute beneficial and desirable biofilm attenuating activity.

### BRIEF DESCRIPTION OF THE INVENTION

Biofilms present a significant health risk to humans and other animals, and are found on a wide range of surfaces ranging from teeth & dental unit water lines, to catheters, medical implants and instruments, to consumer products, and in industrial transportation pipelines & storage containers. Once established, biofilms are extremely difficult to remove, and the microbes that reside within them are much more resistant to conventional antiseptics and antimicrobials than planktonic (free-floating) microbes. While a variety of compositions and methods have been developed for reducing microbial populations, and preventing and removing biofilms, the success of these remains well short of what is desirable. Moreover, while many existing approaches provide some success in terms of biocidal activity, they remain deficient in achieving prevention of biofilm formation and enabling effective and thorough removal of biofilms. Thus, repopulation of residual biofilms with microbes is virtually inevitable. Accordingly, there is a need for surface compositions, composite articles, and methods of treatment that provide robust biofilm attenuating activity to effectively prevent or render biofilms susceptible to removal.

The inventors have surprisingly found that antimicrobial resins, and in some particular embodiments, antibacterial resins, as disclosed herein inhibit initial biofilm formation and effectively disrupt further development of nascent and established biofilms. As further described herein, the activity of compositions and materials according to the disclosure alter the nature of formed biofilms rendering them vulnerable to modest mechanical forces, the alterations including disruption of native biofilm structure. These effects are quantitatively significant, and cause 50% more reduction in total biomass of the biofilm as compared to a control surface. Strikingly, it was further discovered that biofilms developed on the surface of such antibacterial composites are structurally quite different from those grown on control surfaces, and are much more easily removed, as evidenced by the complete removal under relatively low shear force. This is particularly notable in comparison with control biofilm, for which removal could not be achieved even under increasing shear force.

Disclosed herein are compositions, including resins, coatings and articles of manufacture, and methods of making and using the same, the inventions being particularly useful for inhibiting biofilms and enabling their effective removal. The compositions disclosed herein include novel polymeric resins and monomeric non-polymerizable and polymerizable resins.

More specifically, the compositions include, in some embodiments, non-polymerizable antimicrobial mixtures containing a combination of
a) at least one antimicrobially active quaternary ammonium compound, and
b) at least one antimicrobially active quaternary phosphonium compound.
wherein, the combination of components a) and b) are present in a ratio by weight from 1:9 to 9:1.

And wherein the antimicrobially active quaternary ammonium compounds, including imidazolium, ammonium, pyrrolidinium, etc. (component a)) are represented by the formula

[R—N⁺R₁R₂R₃]X⁻ (I)

in which R, R₁, R₂, and R₃ are a preferably straight-chain or branched or cyclic of C2-C20 alkyl radical as same or different length independently; also be as fused cyclic or aromatic ring such as aziridine, azirine, oxaziridine, diazirine, azetidine, azete, diazetidine, pyrrolidine, pyrrole, imidazolidine, imidazole, pyrazolidine, pyrazole, thiazolidine, thiazole, isothioazolidine, isothiazole, piperdine, pyridine, piperzine, diazine, morpholinem oxazine, thiomopholine, thiazine, triazine, triazoles, furanzan, oxadiazole, thiadizole, dithozole, tetrazole, azepane, azepine, diazepine, thiazepine, azocane, azocine, azonane, azonine, etc.

where X⁻ is a counter anion, which can be inorganic, anions (Cl⁻, AlCl₄⁻, PF₆⁻, BF₄⁻, NTf₂⁻/trifluoromethanesulfonyl, DCA⁻/dicyanamide, etc.) or organic anions (CH₃COO⁻, CH₃SO₃⁻, etc.). These quaternary ammonium compounds can be present in the mixtures according to the invention either individually or in admixture with one another.

And wherein antimicrobially active quaternary phosphonium compounds (component b)) are, in particular, compounds corresponding to the following formula

[RP⁺R₁R₂R₃]Y⁻ (II)

in which R, R₁, R₂, and R₃ are a preferably straight-chain, branched or cyclic of C2-C20 alkyl radical as same or different length independently;

Y⁻ is a counter anion, which can be inorganic, anions (Cl⁻, AlCl₄⁻, PF₆⁻, BF₄⁻, NTf₂⁻/trifluoromethanesulfonyl. DCA⁻/dicyanamide, etc.) or organic anions (CH₃COO⁻, CH₃SO₃⁻, etc.).

Or according to the formula

[(R′)₃P⁺R″]Y⁻ (III)

in which R′ is a C1-C5 alkyl radical, a C1-C6 hydroxyalkyl radical or a phenyl radical, R″ is a C3-C18 alkyl radical and Y⁻ is a halide anion, more especially a chloride anion or a bromide anion. The radicals R′ and R″ in formula III are preferably straight-chain or branched or cyclic radicals. The quaternary phosphonium compounds can be present in the mixtures of the invention either individually or in admixture with one another. Examples of quaternary phosphonium compounds of the above type are trimethyl-n-dodecyl phosphonium chloride, triethyl-n-decyl phosphonium bromide, tri-n-propyl-n-tetradecyl phosphonium chloride, trimethylol-n-hexadecyl phosphonium chloride, tri-n-butyl-n-decyl phosphonium chloride, tri-n-butyl-n-dodecyl phosphonium bromide, tri-n-butyl-n-tetradecyl phosphonium chloride, tri-n-butyl-n-hexadecyl phosphonium bromide, tri-n-hexyl-n-decylphosphonium chloride, triphenyl-n-dodecyl phosphonium chloride, triphenyl-n-tetradecyl phosphonium bromide and triphenyl-n-octadecyl phosphonium chloride. Tri-n-butyl-n-tetradecyl phosphonium chloride is preferred.

The compositions also include, in other embodiments, polymerizable antimicrobial mixtures containing at least one type of moiety as defined in I, II, III, the moieties further comprising at least one polymerizable group such as, but not limited to, acrylate, methacrylate, acrylamide, vinyl, vinyl-ether, cyclic ether(epoxy) or cyclic amines and cyclic imine, of which presented as modified R, R₁, R₂, R₃, R′, and R″.

These quaternary ammonium and phosphonium compounds can be present in the mixtures according to the invention either individually or in admixture with one another.

Monomeric and polymeric resins as disclosed herein may be composed of, in some embodiments, the functional non-polymerizable resins containing at least one of each of antimicrobially active quaternary ammonium and phosphonium compounds, and in other embodiments polymerizable resins containing at least one of antimicrobially active quaternary ammonium and phosphonium compounds at least one polymerizable group, wherein according to the various embodiments, the antimicrobially active quaternary ammonium and phosphonium compounds are present in compositions, articles and coatings in amounts of from about 0.1 weight percent to about 10 weight percent, the amount selected to achieve balanced biofilm attenuating activity, antibacterial activity/microbial cytotoxicity and mechanical properties of the compositions, articles and coatings. Thus, in some embodiments, the antimicrobially active quaternary ammonium and phosphonium compounds are present in amounts from about 0.1 weight percent to about 10 weight percent, and in some embodiments up to 50 weight percent or more, including 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 20.0, 30.0, 40.0 and 50.0 and fractional increments there between.

In accordance with various embodiments, the monomers and polymeric resins described herein that are useful for interrupting biofilm formation are useful in a variety of applications whereby they may be formed into solid articles, applied as solid or film coatings on the surfaces of solid articles, or dispersed on, in or throughout other resins and composites, or coated on or dispersed in small particles that are used in fluid suspensions or in filtration, and they may be dispersed free in fluid suspensions. Accordingly, in various alternate embodiments, the monomeric and polymeric resins include, broadly, articles of manufacture, components, reagents, and kits.

Other features and advantages of the present invention will be apparent from the following more detailed description, taken in conjunction with the accompanying drawings which illustrate, by way of example, the principles of the invention.

### BACKGROUND OF THE INVENTION

Antimicrobial/Antibacterial Agents:

There are inorganic and organic compounds that have been used as Antimicrobial/antibacterial Agents or Plaque Inhibitory Agents. They can be in solid or liquid form; in charged or neutral state, leachable or non-leachable/immobilized, synthesized or naturally-occurred/extracted from plants, etc.

Control of oral biofilms is essential for maintaining oral health and preventing dental caries, gingivitis and periodontitis. However, oral biofilms are not easily controlled by mechanical interventions and represent difficult targets for chemical control. Here are some of the widely used Antimicrobial/antibacterial Agents in dentistry and their action mechanisms respectively.

Amine Alcohol:

Example: Octapinol, Delmopinol:

Actions: plaque inhibition via interfering with plaque matrix formation and reducing bacterial adherence:

Bisguanides:

Example: Chlorhexidine (CHX), Alexidine, Octenidine, Polyhexamethylene (PHMB)

Actions: Antibacterial, bacterial cell wall damage, plaque inhibition by binding to bacteria cell membrane.

Chlorhexidine digluconate is the most studied, which is effective against both Gram-positive and Gram-negative bacteria including aerobes and anaerobes and yeasts and fungi. CHX is powerful antimicrobial agent—it's able to bind to a variety of substrates while maintaining its antibacterial activity. It is then slowly released, leading to the persistence of effective concentrations. Although two salts of CHX have similar antibacterial activity, the diacetate and dihydrochloride, the diacetate was more soluble. High concentration of CHX nearly eliminates all microbial cells and it is not beneficial towards maintaining a healthy microbiota balance in oral biofilm. Successful antimicrobial agents are able to maintain the oral biofilm at levels compatible with good oral health but without disrupting the natural and beneficial properties of the resident oral microflora.

Enzymes:

Example: Lactoperoxidase, Lysozyme, Glucose oxidase, Amyloglucosidase

Actions: Antibacterial, enhanced host defense mechanisms

Essential Oil:

Example: Thymol, Eucalyptol,

Actions: Antibacterial, Antioxidative activity, inhibition of enzyme activity, reducing glycolysis, reducing bacterial adherence

Oxygenating Agents:

Example: Hydrogen peroxide, sodium peroxycarborate

Actions: Antibacterial

Fluorides:

Example: Sodium fluorides, stannous fluoride, amine fluoride, monofluorophosphate:

Actions: prevents demineralization, enhances remineralization, antibacterial effect derived from non-fluoride portion

Metal Ions:

Example: Stannous, Zinc, Silver, Copper:

Actions: Antibacterial, Plaque inhibition, inhibiting enzyme systems and glycolysis

Plant Extracts/Natural Products:

Example: Sanguinarine extracts

Actions: Antibacterial, Plaque inhibition by suppression growth of bacterial strains and enzyme activity

Phenols:

Example: Triclosan: Antibacterial,

Actions: plaque inhibition, interfering with plaque metabolism, disruption of bacterial cell

Quaternary ammonium (QAS) and/or phosphonium salts (QPS):

Example:

Cetylpyridinium chloride (CPC). Moderate plaque inhibitory activity. Although they have greater initial oral retention and equivalent antibacterial activity to CHX, they are less effective in inhibiting plaque and preventing gingivitis.

Cetyltrimethylammonium bromide

Tetradecyldimethylbenzylammonium chloride

Benzethonium chloride

Methylbenzethonium chloride

Undecoylium chloride

p-tert-Octylphenoxyethoxyethyldimethylbenzyl ammonium chloride

Actions: Antibacterial, plaque inhibition by interaction with microorganism

Quaternary ammonium salts are frequently used as antibacterial agents that disrupt cell membranes through the binding of their ammonium cations to anionic sites in the outer layer of bacteria.

Surfactants:

Example: Sodium lauryl sulphate

Actions: Antibacterial, inactivated bacterial enzymes

Bacteriophages:

Inhibitors of the biosynthesis of fatty adds:

Antimicrobial peptides:

Chelating agents: Ethylene glycol tetraacetic acid (EGTA) and trisodium citrate (TSC)

Actions: Metallic cations such as Mg²⁺ and Ca²⁺ can also affect bacterial growth and biofilm formation. These divalent cations can stimulate cell-cell adhesion and aggregation through their interactions with cell-wall teichoic acids. Therefore, removal of free cations from the environment reduces intercellular adhesion and subsequent biofilm formation.

Nanoparticles: Nanosilver, QAS modified nanofillers, QAS modified nanogels

nano-sized metals and metal oxides, mainly silver (Ag), titanium dioxide(TiO₂), zinc oxide (ZnO) and cooper II oxide (CuO)

Antimicrobial polymers, also known as polymeric biocides, is a class of polymers with antimicrobial activity, or the ability to inhibit the growth of microorganisms such as bacteria, fungi or protozoans, such as quaternary ammonium poly(ethylene imine)(QA-PEI) nanoparticles.

Antimicrobial Monomers and Polymers

This synthetic method involves covalently linking antimicrobial agents that contain functional groups with high antimicrobial activity, such as hydroxyl, carboxyl, or amino groups to a variety of polymerizable derivatives, or monomers before polymerization. The antimicrobial activity of the active agent may be either reduced or enhanced by polymerization. This depends on how the agent kills bacteria, either by depleting the bacterial food supply or through bacterial membrane disruption and the kind of monomer used. Differences have been reported when homopolymers are compared to copolymers.

In order for an antimicrobial polymer to be a viable option for large-scale distribution and use there are several basic requirements that must be first fulfilled:

The synthesis of the polymer should be easy and relatively inexpensive. To be produced on an industrial scale the synthetic route should ideally utilize techniques that have already been well developed.

The polymer should have a long shelf life, or be stable over long periods of time. It should be able to be stored at the temperature for which it is intended for use.

If the polymer is to be used for the disinfection of water, then it should be insoluble in water to prevent toxicity issues (as is the case with some current small molecule antimicrobial agents).

The polymer should not decompose during use, or emit toxic residues.

The polymer should not be toxic or irritating to those during handling.

Antimicrobial activity should be able to be regenerated upon loss of activity.

Antimicrobial polymers should be biocidal to a broad range of pathogenic microorganisms in brief times of contact.

TABLE 1
Antimicrobial Monomers, and Polymers Synthesized from Antimicrobial
Monomers and their Antimicrobial Properties

| | Inhibited | | Comparison of |
|---|---|---|---|
| | Microbial | Antimicrobial | Polymers with |
| Monomer | Species | Mechanism | Monomer |
| 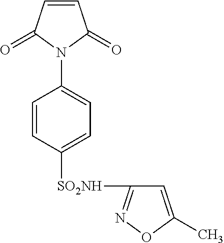 | Fungi: C. albicans; A. niger | Slow release of 4- amino-N-(5- methyl-3- isoxazoly)benzen esulfonamide | The homopolymer is more effective than the monomer at all concentrations.^{[6]} |
| 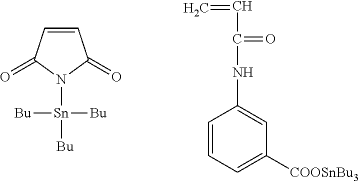 | Bacteria: Gram- positive; Gram- negative | Tin moiety on the polymer surface interacts with the cell wall. | Copolymerization of antimicrobial monomer and styrene decreases the potency of the monomer.^{[7]} |
| 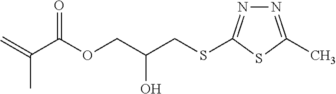 [SMILES: C=C(C)C(=O)OCC(O)CSc1nnc(C)s1] | Bacteria: S. aureus; P. aeruginosa; E. coli; | The presence of benzimidazole derivatives inhibit cytochrome P-450 monooxygenase | The homopolymer is more effective than the monomer.^{[8]} |
| 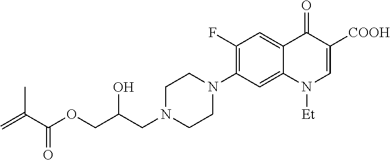 [SMILES: C=C(C)C(=O)OCC(O)CN1CCN(c2cc3c(cc2F)c(=O)c(C(=O)O)cn3CC)CC1] | Bacteria: Gram- positive; Gram- negative | Release of norfloxacin which inhibits bacterial DNA gyrase and cell growth.^{[9]} | — |
| 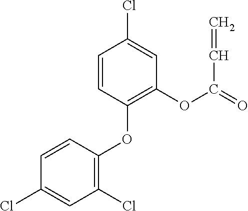 [SMILES: C=CC(=O)Oc1cc(Cl)ccc1Oc1ccc(Cl)cc1Cl] | Bacteria: P. aeruginosa; Staphylococcus spp. | Active agent is 2,4,4′-trichloro- 2′- hydroxydiphenyl- ether | The homopolymer and copolymers with methyl methacrylate, styrene are all less effective than the monomer.^{[10]} |
| 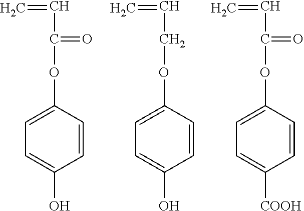 | Bacteria: S. aureus; P. aeruginosa | Active agent is phenol group. | Polymerization significantly decreases the anitimicriobial activity of the monomers.^{[11]} |
| 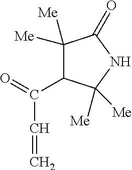 | Bacteria: E. coli | Direct transfer of oxidative halogen from polymer to the cell wall of the organism.[12] | — |
| 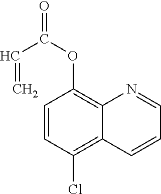 [SMILES: C=CC(=O)Oc1ccc(Cl)c2cccnc12] | Bacteria: E. coli; S. aureus; S. typhimurium | Release of 8- hydroxyquinoline moieties | The homopolymer and the copolymers with acrylamide are both less effective than the monomer.^{[13]} |
| 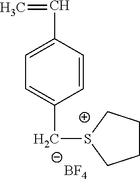 | Bacteria: Gram- positive bacteria | Active agent is Sulfonium salt | The homopolymer is more effective than the corresponding model compound (p- ethylbenzyl tetramethylene sulforium tetrafluoroborate). |
| 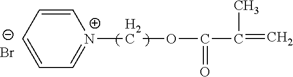 | Bacteria: Oral Streptococci spp. | Direct cationic binding to cell wall, which leads to the disruption of the cell wall and cell death.^{[15]} | — |
| 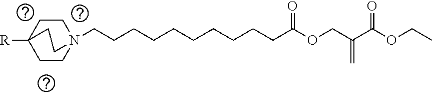 | Bacteria: S. aureus; E. coli | Cationic biocides targets the cytoplasmic membranes; Similarities of the polymer pendent | The monomers are not active, while homopolymers show moderate activities in concerntration from 1 mg/mL to 3.9 mg/mL. |
| | | groups and the lipid | |
| | | layer enhances | |
| | | diffusion into the cell | |
| | | wall | |
| 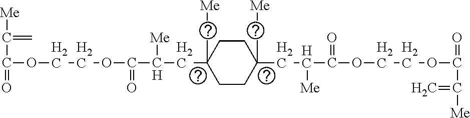 [SMILES: C=C(C)C(=O)OCCOC(=O)C(C)CC1(CC)CCC(CC)(CC(C)C(=O)OCCOC(=O)C(=C)C)CC1] | Bacteria: S. aureus; E. coli | Membrane disruption | — |
| 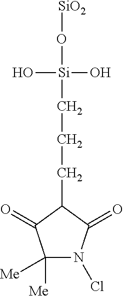 | Bacteria; Staphylococcus spp.;E. coli | Immobilization of high concentrations of chlorine to enable rapid biocidal activities and the liberation of very low amounts of corrosive free chlorine into water | — |

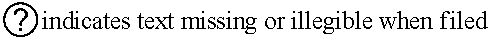

TABLE 2
Antimicrobial Polymers Synthesized from Preformed Polymers and Antimicrobial
Properties

| | Inhibited Microbial | Antimicrobial |
|---|---|---|
| Polymer | Species | Mechanism |
| 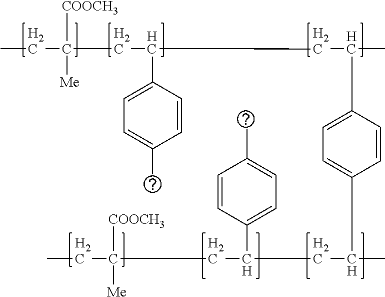 | Fungi: C. albicans; A. flavus; Bacteria: S. aureus; E. coli; B. subtilis; F. oxysporum | Active group: Phosphonium groups. |
| 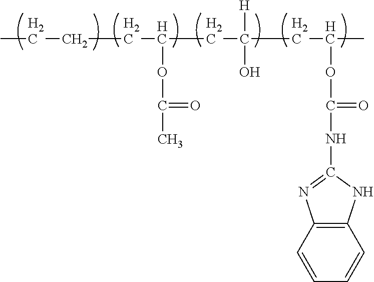 | Fungi: A. fumigatus; P. pinophilum | The release of m-2- benzimidazole- carbamoyl moiety. |
| 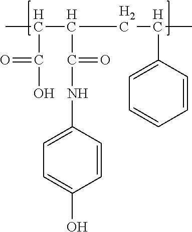 | Bacteria: E. coli; S. aureus | Active groups: phenolic hydroxyl group. |
| 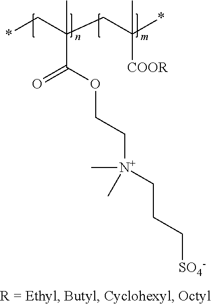 | Bacteria: E. coli; S. aureus | Active group: Quaternary ammonium group. |
| 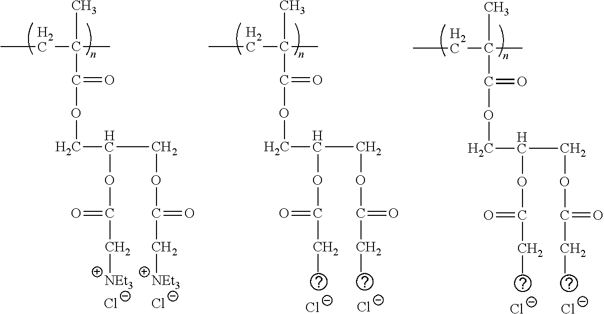 | Fungi: T. rubrum; Bacteria: Gram-negative bacteria | Active groups: Phosphonium and quaternary ammonium groups. |

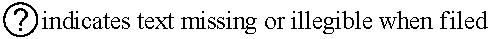

Chitin is the second-most abundant biopolymer in nature. The deacetylated product of chitin-chitosan has been found to have antimicrobial activity without toxicity to humans. This synthetic technique involves making chitosan derivatives to obtain better antimicrobial activity. Currently, work has involved the introduction of alkyl groups to the amine groups to make quaternized N-alkyl chitosan derivatives, introduction of extra quaternary ammonium grafts to the chitosan, and modification with phenolic hydroxyl moieties.

This method involves using chemical reactions to incorporate antimicrobial agents into the polymeric backbones. Polymers with biologically active groups, such as polyamides, polyesters, and polyurethanes are desirable as they may be hydrolyzed to active drugs and small innocuous molecules. For example, a series of polyketones have been synthesized and studied, which show an inhibitory effect on the growth of B. subtilis and P. fluorescens as well as fungi, A. niger and T. viride.

Bacterial Species, Particularly Oral

The human mouth is home to numerous colonies of microorganisms. While most of these oral bacteria do no harm, there are other species in the mix that are disease causing and can affect health.

Over 700 different strains of bacteria have been detected in the human mouth, though most people are only host to 34 to 72 different varieties. Most of these bacterial species appear to be harmless when it comes to health. Others, known as probiotics, are beneficial bacteria that aid in the digestion of foods. Other bacteria actually protect teeth and gums. There are some bacteria, however, that we'd rather do without, since they cause tooth decay and gum disease.

There is a distinctive bacterial flora in the healthy oral cavity which are different from those that cause oral disease. For example, many species specifically associated with periodontal disease, such as P. gingivalis, T. forsythia, and T. denticola, were not detected in any sites tested. In addition, the bacterial flora commonly thought to be involved in dental caries and deep dentin cavities, represented by S. mutans, Lactobacillus spp., Bifidobacterium spp., and Atopobium spp., were not detected in supragingival and subgingival plaques from clinically healthy teeth.

Many of these bacterial species, over 50% have not been cultivated; have been detected in the oral cavity. The oral cavity is comprised of many surfaces, each coated with a plethora of bacteria, the proverbial bacterial biofilm. Some of these bacteria have been implicated in oral diseases such as caries and periodontitis, which are among the most common bacterial infections in humans. In addition, specific oral bacterial species have been implicated in several systemic diseases, such as bacterial endocarditis, aspiration pneumonia, osteomyelitis in children, preterm low birth weight, and cardiovascular disease.

Normal Oral Bacterial Flora in Healthy Subjects:

FIG. 1: Site Specificity of Predominant Bacterial Species in the Oral Cavity. In general, bacterial species were selected on the basis of their detection in multiple subjects for a given site. Distributions of bacterial species in oral sites among subjects are indicated by the columns of boxes to the right of the tree as follows: not detected in any subject (clear box), <15% of the total number of clones assayed (yellow box), >15% of the total number of clones assayed (green box). The 15% cutoff for low and high abundance was chosen arbitrarily. Marker bar represents a 10% difference in nucleotide sequences.

TABLE 3
Number of predominant bacterial species per site and subject

| | Total no. of species/site | | | | | | | | | |
|---|---|---|---|---|---|---|---|---|---|---|
| Subject | | Maxillary | Tongue | Tongue | Hard | Soft | | Tooth | | Total no. of |
| no. | Buccal | vestibule | dorsum | lateral | palate | palate | Tonsils | surface | Subgingival | species/subject |
| 1 | 20 | 5 | 23 | 14 | 21 | 16 | 28 | 27 | 14 | 66 |
| 2 | 12 | 6 | 13 | 18 | 18 | 20 | 14 | 12 | 6 | 45 |
| 3 | 11 | 9 | 10 | 9 | 15 | 14 | 22 | 21 | 22 | 72 |
| 4 | 5 | 7 | 10 | 8 | 4 | 6 | 11 | 12 | 4 | 34 |
| 5 | 4 | 3 | 17 | 20 | 6 | 13 | 18 | 16 | 21 | 64 |
| Total | 32 | 15 | 40 | 34 | 42 | 38 | 59 | 52 | 47 | |

Table 3 and FIG. 1 represent the overall summary showing that there are emerging bacterial profiles that help define the healthy oral cavity. As observed, several species, such as S. mitis and G. adiacens, were detected in most or all oral sites, whereas several species were site specific. For example, R. dentocariosa, Actinomyces spp., S. sanguinis, S. gordonii, and A. defectiva appeared to preferentially colonize the teeth, while S. salivarius was found mostly on the tongue dorsum. Some species appeared to have a predilection for soft tissue, e.g., S. sanguinis and S. australis did not colonize the teeth or subgingival crevice. S. intermedius preferentially colonized the subgingival plaque in most of the subjects but was not detected in most other sites. On the other hand, Neisseria spp. were not found in subgingival plaque but were present in most other sites. Simonsiella muelleri colonized only the hard palate. Indeed, S. muelleri was initially isolated from the human hard palate, although it has been isolated from a neonate with a dental cyst and early eruption of teeth. Several Prevotella species were detected in most sites, but only in one or two subjects. For example, P. melaninogenica and Prevotella sp. clone BE073 were abundant in seven out of nine sites of one subject and were detected sporadically in other subjects. Prevotella sp. clone HF050 was found in the maxillary anterior vestibule of one subject, dominating the bacterial flora as 44% of the clones. This clone was also found in lower proportions on the soft palate and tonsils of another subject.

Microbial Biofilm Composiryion of Oral Disease State

Many studies have been performed that attempt to determine which bacterial species are directly involved in oral pathology. Because many of the plaque-mediated oral diseases occur at regions already containing an extremely diverse microflora, it is difficult to exactly specify which of these species are pathogenic. Additionally, the bacterial traits associated with carcinogenicity (acid production, acid tolerance, intracellular and extracellular polysaccharide production) point to more than a single bacterial species. We do know, however, that many of the desirable bacterial species involved in healthy plaque biofilms include Streptococcus sanguis, S. gordonii, S. oralis, and the Actinomyces species, in addition to other related bacteria with a low acid tolerance. Therefore, it seems that healthy dental biofilm microflora consist of species with limited tolerance for acid, as bacteria involved in the formation of dental caries are those with a very high acid tolerance.

The Two Most Common Harmful Bacteria

S.s mutans is the bacteria that lives in the mouth of animal hosts, in particular, human hosts and feeds on the sugars and starches consumed by a host. That alone would not be so bad, but as a by-product of its ravenous appetite, it produces enamel-eroding acids, which make S. mutans the main cause of tooth decay in humans.

P. gingivalis is usually not present in a healthy mouth, but when it does appear, it has been strongly linked to periodontitis. Periodontitis is a serious and progressive disease that affects the tissues and the alveolar bone that support the teeth. It is not a disease to be taken lightly. It can cause significant dental pain, inflammation and can eventually lead to tooth loss and bone loss. Moreover, ample investigations and studies have reported the correlation between the periodontitis and heart/cardiovascular disease (CVD), i.e. periodontitis can be a risk factor for heart disease.

Caries: Despite the lack of exact knowledge on every pathogen involved in caries production, the factors responsible for microbial homeostasis within a biofilm are known and recognized. The initial change in environment is due to an increased amount of fermentable carbohydrates in the diet of the host. The anaerobic, acid-producing bacteria present in the plaque biofilm thus produce an increased amount of acid due to fermentation, consequently lowering the pH of the biofilm. When the pH drops, there is an increase in these acid-tolerant bacteria, as they are the only ones that can survive and perform glycolysis in such acidic environments. Some of the more common bacterial species responsible for this include Streptococcus mutans, S. sorbrinus, and Lactobacillus casei which can perform glycolysis at a pH level as low as 3.0. A select number of bacteria involved in the dental plaque biofilm shows the vast differences that exist between normal oral microflora species. At this acidic pH, the highly acid-tolerant bacterial biofilm is capable of demineralizing the tooth enamel, with greater degrees of acidity causing faster rates of demineralization. Of course, this acidification is originally caused by sugar ingestion, meaning if sugar intake stops, the pH value of the biofilm will rise again and remineralization of the enamel can occur. Caries will result, however, if the acidification-demineralization phase is more damaging and more frequent than the alkalinization-remineralization phase can manage to fix the damage. The demineralization of tooth enamel can also occur solely from the presence of highly acidic substances in the oral cavity. This is why people who drink excessive amounts of sports drink or soda pop (with a highly acidic pH of 2.34.4) have a much higher prevalence of caries. In short, when sugar is ingested and acid is produced as a metabolic byproduct, bacteria that can survive in these acidic environments will thrive. These are the key pathogens to caries production. It is important to recognize, however, that some of the known bacterial species involved in caries production, like S. mutans, are also present in healthy plaque biofilm. in addition to also being absent from some sites of caries production. Thus, we know that there is not one particular bacterial species responsible for caries production, but a collection of several, exhibiting similar characteristics.

Periodontitis and Peri-implantitis: Some level of periodontal disease affects a majority of the adult population of the United States. Because of this, it is of great importance to the medical and dental community and can thus be considered a public health problem. Periodontitis, if not treated early, can lead to alveolar bone and tooth loss. It is defined by deep pockets formed between the tooth surface and the gum, with this deep pocket being easily colonized by microbes due to the small dentinal tubules and enamel fissures that lead directly into the gums from the open space of the mouth. This area is incredibly difficult to reach via typical oral health care mechanisms (tooth brush, floss, etc.), which often leads to the diseased states of gingivitis or periodontitis, that have varying levels of severity. Also, it should not be neglected that periodontitis can be a risk factor for heart disease.

Much of the microflora existing in these deep periodontal pockets are gram-negative anaerobes, with a very diverse population of spirochetes. In the early stages of periodontal disease, known as gingivitis, the initial microbial colonization of the plaque biofilm seems to involve members of the yellow, green and purple “clusters”. Secondary colonization occurs with members of the orange and red clusters, and these become more dominant. The increased levels of the red and orange cluster bacteria lead to proliferation by members of all the original and secondary colonizing species. At a certain point, the organisms must disperse to other locations within the oral cavity to ensure survival. As shown in FIG. 2, a study found that spirochetes and P. gingivalis were more prevalent in diseased sites of diseased patients than in healthy sites of diseased patients. It was also found that the organisms were found more frequently in healthy sites of diseased patients than in healthy sites of healthy patients, which is evidence for the dispersal mechanism previously mentioned.

Microbial Complexes Arranged into Clusters

As periodontal disease gets more severe, checkerboard DNA-DNA hybridization experiments have been performed to give a better idea of the species involved in periodontitis. This molecular biology experiment was used to detect the presence of various bacterial species by using known DNA probes on the horizontal lanes, and plaque samples from a number of patients in the vertical lanes. By looking at the blot, it is clear which bacterial species were present in the plaque in these periodontal patients, as the DNA probes bound to their corresponding DNA sequences of bacteria present in the plaque. Further analysis has been performed from this, to show that the most prevalent bacterial species involved in periodontitis is Actinomyces naeslundii. These tests were performed on 40 species of which there were known molecular probes. Unfortunately, there is no way to determine every bacterial species involved in the plaque biofilm of periodontitis patients, as a molecular probe is needed for checkerboard DNA-DNA hybridization, and probes have not been developed for all species.

Peri-implantitis is very similar to periodontitis, however, it does differ in some aspects. Because dental implants are not surrounded by periodontal ligaments, they have differing biomechanics and defensive cell-recruitment. Peri-implantitis refers to the destruction of the supporting peri-implant tissue due to a microbial infection. These infections tend to occur around places where residual teeth or failing implants can act as reservoirs for bacteria and form biofilm colonies. Interestingly, the bacterial species involved in peri-implantitis are very similar to those that play a key role in periodontitis. The two diseases differ in some key ways, but they do have many similarities and research in both can help lead to better treatment and prevention.

Dental plaque biofilms are a diverse, functioning microbial community that is found on every organism on earth that has teeth. Because of the wide diversity of organisms involved in the development and proper function of plaque biofilm, it is difficult to know everything there is to know about these fascinating microbial communities. These biofilms employ a great deal of inter-cell communication to not only keep themselves alive, but also to protect the host. Their existence, while involved in many pathogenic oral diseases, is of much benefit to the host at the early stages of development, as it provides the teeth with a layer of protection that cannot be matched. In time, we will continue to discover more about the biochemical and developmental functions involved in dental plaque biofilms, which can help us to not only learn about microbiology, but also to improve oral health, which is of great importance to our overall well-being.

FIG. 3 shows a representative sample of human host subjects levels of microbes in dental plaque. Checkerboard DNA-DNA hybridization analysis was employed to detect the presence of 40 microbial species in 28 subgingival plaque biofilm samples in a group of host subjects.

In addition to dentistry, antibacterial is also an important branch of functional coating that plays an important role not only for general hygiene but also for saving life as disinfectant in places such as operation theatre in hospitals. Antibacterial studies are mostly evolved around S. aureus, E. coli and P. aeruginosa. S. aureus is frequently found in human respiratory tract and skin. It is a common cause of skin infections, respiratory disease, and food poisoning. On the other hand, E. coli is commonly found in lower intestine of warm blooded organisms. It usually causes the food poisoning and is occasionally responsible for product recalls due to food contamination. The third bacteria P. Aeruginosa is considered as one of the toughest bacterial strain and able to survive in harsh environments.

Biofilm-forming bacteria related to human disease and medical devices (Shadia M. Abdel-Aziz, Aeron A (2014) Bacterial Biofilm: Dispersal and Inhibition Strategies. SAJ Biotechnol 1(1): 105):

TABLE 4
Some human disease associated with bacteria biofilms

| Human Disease | Biofilm-forming Bacteria |
|---|---|
| Cystic fibrosis pneumonia | P. aeruginosa and B. cepacia |
| Meloidosis | P. pseudomallei |
| Necrotizing fasciitis | Group A streptococci |
| Musculoskeletal infections | Staphylococci and other Gram-positive cocci |
| Otitis media | H. influenzae (Non-typable strains) |
| Biliary tract infection | E. coli and other enteric bacteria |
| Urinary catheter cystitis | E. coli and other Gram-negative rods |
| Bacterial prostatitis | E. coli and other Gram-negative bacteria |
| Periodontitis | Gram negative anaerobic oral bacteria |
| Dental caries | Streptococcus spp. And other acidogenic |
| | Gram positive cocci |

TABLE 5
Food-borne pathogens and spoilage bacteria in biofilm

| Growing surface | Food-borne pathogens |
|---|---|
| Dairy processing plant, conveyor belt | L. monocytogenes |
| Drain, vegetable and meat surface | Pseudomonas spp. |
| Pepelie, joint in processing environment, hot fluid | Bacillus spp. |
| Poultry processing environment | Salmonella ssp. |

TABLE 6
Microorganisms associated with biofilm
on indwelling medical devices

| Medical Devices | Causative organism |
|---|---|
| Urinary catheter, Intra-urine device, Prosthetic | Coagulase-negative |
| heart valve, Central venous catheter | Staphylococci |
| Urinary catheter, Central venous catheter | K. pneumoniae |
| Artificial hip prosthesis, Central venous catheter, | P. aeruginosa |
| Intra-urine device | |
| Artificial voice prosthesis, Central venous | C. albicans |
| catheter, Intra-urine device | |
| Artificial hip prosthesis, Central venous | S. aureus |
| catheter, Intra-urine device, Prosthetic heart | |
| valve | |
| Artificial hip prosthesis, Prosthetic heart valve, | Enterococcus spp. |
| Urinary catheter | |

In addition to antimicrobial/antibacterial chemical compounds, photonic & photochemical approaches have also been investigated to modify the composition and metabolic activities of biofilm. Ultraviolet light, particularly UVC (200-280 nm), also shows germicidal effect. Many microbial cells are also highly sensitive to killing by blue light (400-470 nm) due to accumulation of naturally occurring photosensitizers such as porphyrins and flavins. Near infrared light has also been shown to have antimicrobial effects against certain species.

Biofilms

Biofilms are known in the art, and a brief description is provided herein below. Currently there are three types of biofilm control strategies: prevent, kill, removal. Many conventional antimicrobial agents fail to remove biofilm, for example mouth rinse is able to kill bacteria but not remove biofilm. The biofilm removal approach involves in attacking the mechanical integrity of biofilm, targeting biofilm matrix adhesion instead of killing bacteria, such as baking soda to weaken biofilm structure by raising pH 8.2-8.3, and enzymatic treatment or alternative dispersant treatment. The thicker the biofilm is, the harder to remove. Thus an integrated method with biofilm inhibition and biofilm removal should be a promising approach. FIG. 4A provides a visual representation of biofilm treatment and removal.

General strategies to modify or enable active surfaces for biofilm prevention, control, and detachment include the following: surface modification, such as using protein repellant polymer or other anti-adhesion agents; both organic-based & inorganic-based antimicrobial agents; organic-based antimicrobial agents include antibiotics, chlorohexidine, quaternary ammonium monomer, NAC, etc.; inorganic-based antimicrobial Agents such as Silver Nanoparticle (NP), gold NP, zinc oxide, quaternary ammonium nanoparticles (such as quaternary ammonium poly(ethylene imine)(QA-PEI)), TiO₂: glutaldehyde, formaldehyde, etc.; antibiofilm enzyme; anti-microbile peptide; chelating agents such as ethylene glycol tetraacetic acid (EGTA) and trisodium citrate (TSC); ultrasonic treatment; bioelectric treatment; photonic and photochemical treatment; ultraviolet light, particularly UVC (200-280 nm); blue light (400-470 nm) due to accumulation of naturally occurring photosensitizers such as porphyrins and flavins; near infrared light.

### BRIEF DESCRIPTION OF THE DRAWINGS

FIG. 1 shows graphical results for Site Specificity of Predominant Bacterial Species in the Oral Cavity.

FIG. 2 shows graphical results for spirochetes and P. gingivalis analyses.

FIG. 3 shows a representative sample of human host subject levels of microbes in dental plaque.

FIG. 4A provides a visual representation of biofilm treatment and removal.

FIG. 4B provides the chemical structures of some exemplary embodiments of compositions according to the disclosure.

FIG. 5 shows a graphic of a Biofilm growth protocol.

FIG. 6 shows show the 3D architecture of 67 h-old biofilms formed on each surface.

FIG. 7 shows the quantitative data of biomass from each surface.

FIG. 8 shows the results of pH analysis of supernatant surrounding test composite and control composite.

FIG. 9 shows images of the supernatant during biofilm growth.

FIG. 10 shows the remained biomass from each composite surface after applying shear stress (n>=12).

FIG. 11 shows the representative confocal image of 67 h biofilms after exposure to shear stress of 0.804 N/m².

FIG. 12 shows EPS-matrix in 2D Cartesian coordinate system (XY, YZ, and XZ planes)

FIG. 13 shows projection image of skeletonized EPS-matrix.

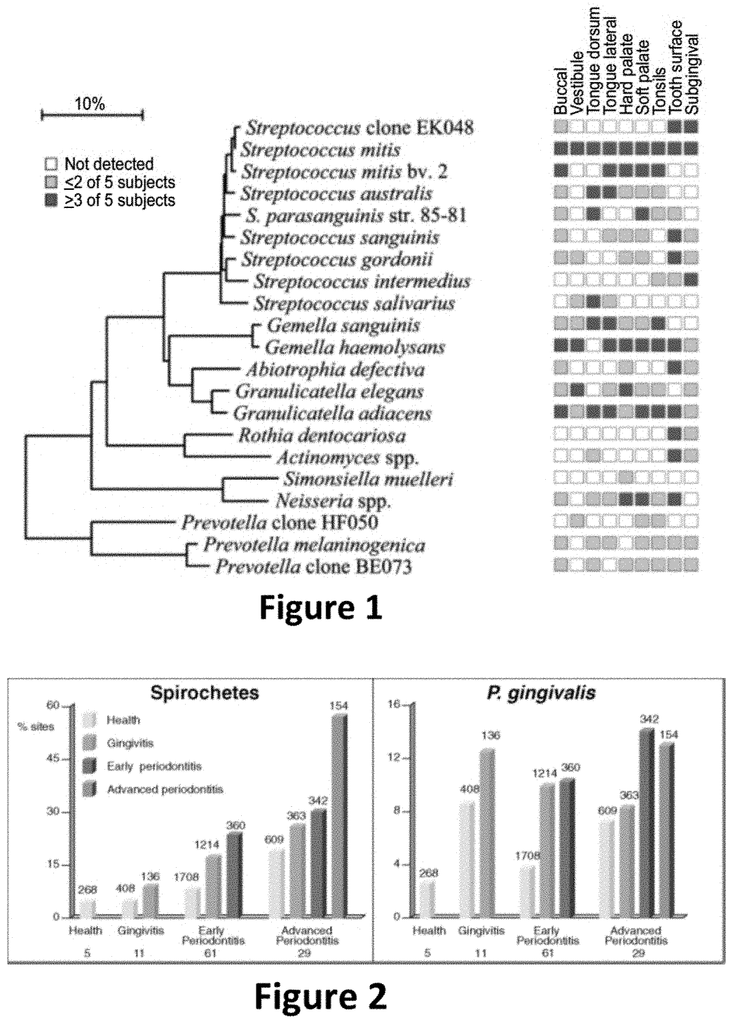

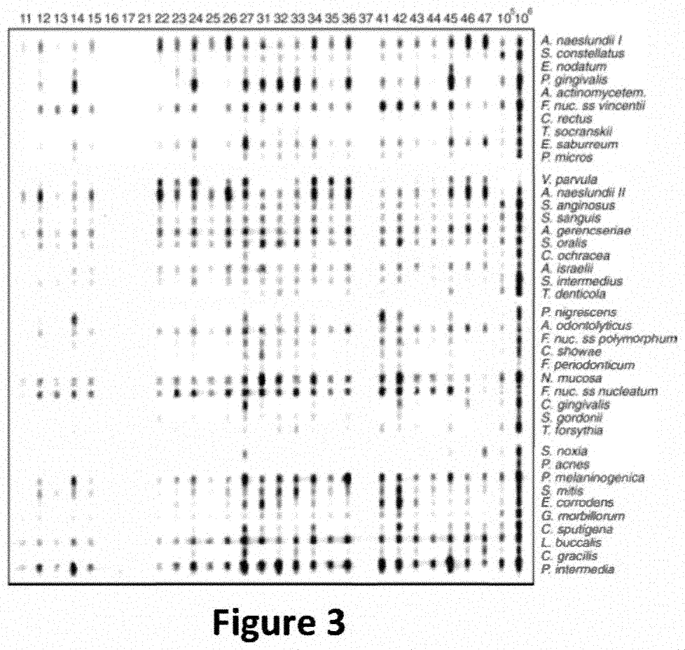

Wherever possible, the same reference numbers will be used throughout the drawings to represent the same parts.

### DETAILED DESCRIPTION OF THE INVENTION

This description provides exemplary embodiments in accordance with the general inventive concepts and is not intended to limit the scope of the invention in any way. Indeed, the invention as described herein is broader than and not intended to be limited by the exemplary embodiments, drawing set forth herein, and the terms as used herein have their full ordinary meaning and as described herein.

As described herein in the examples in the context of in vitro study on biofilm formation, development and detachment, the inventors unexpectedly discovered that S. mutans biofilm on the surface of an antibacterial composite according to the disclosure was significantly reduced in comparison to a control composite and hydroxyapatite (HA). It was further discovered that the mechanical stability of the S. mutans biofilm formed on such antibacterial surface was significantly disrupted as evidenced by complete removal of the biofilm with moderate shear force from the inventive composite. In contrast, the biofilm formed on the control composite and the HA proved to be not susceptible to removal.

Here it is disclosed an effective methodology to remove biofilms in general. Active surfaces could effectively inhibit not only the initial biofilm formation but also further biofilm development. Total biomass formed on such active surfaces would be significantly reduced at least by 50%. The mechanical stability of the biofilm formed on such active surfaces could be significantly weakened and much less effort could be needed for a complete removal with moderate shear force as applied by a tooth brush, water jet, or ultrasonic treatment.

In accordance with various embodiments, such active surfaces could be formed in bulk from compositions formulated with a variety of antibacterial/antimicrobial components, including but not limited to polymerizable resins or additives, non-polymerizable additives, or particles/fillers or a combination of both.

In accordance with some embodiments, such active surfaces could be formed into a coating with a range of thicknesses from compositions formulated with a variety of antibacterial/antimicrobial components, including but not limited to polymerizable resins or additives, non-polymerizable additives, or particles/fillers or a combination of both.

In accordance with some embodiments, the antibacterial/antimicrobial components could be non-cleavable for long-lasting effectiveness.

In accordance with the various embodiments, the antibacterial/antimicrobial components will be loaded in a final composition of 0.1-10% wt/wt or more and up to 50% wt percent for balanced antibacterial activity, cytotoxicity and mechanical property.

In accordance with some embodiments, articles of manufacture, composite articles and materials and coated surfaces comprising any one or more of the non-polymerizable and polymerizable mixtures of quaternary ammonium and phosphonium compounds can be reactivated chemically or by abrasion/heating or other treatment after a period of wear or exposure to fluids or other materials that may comprise microbes. These are non-leachable components and thus it is expected that such an active surface can be readily regenerated as needed.

In some embodiments, the compositions are formulated for providing one or more of coating onto, infusion into, dispersion within, or formation of articles of manufacture for Dental Composite, Dental Adhesive, Dental Cement, Dental Sealant. Dental Liner, Dental Varnish, Denture, Root Canal Sealer, Implant Cement, Orthodontic Cement, Self-disinfected Dental Impression Material, Wearable or removable dental plaque treatment device (Antibacterial Night Guard). According to such embodiments, the compositions can be used in Resin Composite-based CAD/CAM Blocks: for Temporary Crown-bridge Composite; for Pediatric Crown; for Esthetic Orthodontic Aligner; for Esthetic Polymer based Orthodontic Bracket (and coating for metal/ceramic bracket): and in some particular embodiments, the compositions can be used in Coating for Dental Implant Abutment. And according to other such embodiments, the compositions may be provided in suspension or coated on micro or nanoparticles for use in mouthwashes, dental strips, dental films and gels, toothpaste and other dental care items.

Such an active surface can be readily formed on top of any non-active bulk substrates, metal, polymer or ceramic, etc., in a form of coating to cover such a non-active material to generate an active surface accordingly.

In other embodiments, the compositions are formulated for providing one or more of coating onto, infusion into, dispersion within, or formation of articles of manufacture for medical and personal care applications, including continuous positive airway pressure (CPAP) device, Ventilation equipment, Central lines, Kwires and screws for fracture fixation, and orthopedic reduction or distraction and other medical implants, catheters, intravascular catheters, dialysis shunts, wound drainage tubes, skin sutures, vascular grafts, implantable meshes, intraocular devices, heart valves, graft materials, needles, transdermal and transmucosal patches, sponges, and personal care and hygiene products selected from but not limited to tampons, sponges, intrauterine devices, diaphragms, condoms, gloves, drapes and films, wound dressings, tapes and dressings, and the like.

In yet other embodiments, the compositions are formulated for providing one or more of coating onto, infusion into, dispersion within, or formation of articles of manufacture for the inner surface of oil pipelines for reduced biofilm build-up, and likewise for containment and shipping vessels for oil and petrochemical products generally. In other examples, the compositions are formulated for use in connection with storage and shipment of paints and other organic based materials for domestic and/or industrial use. In certain embodiments, the compositions may provide protective effects for reducing rust and general degradation of metal storage and transport materials, and likewise for containment and shipping vessels for oil and petrochemical products generally. In other examples, the compositions are formulated for use in connection with storage and shipment of paints and other organic based materials for domestic and/or industrial use. In certain embodiments, the compositions may provide protective effects for reducing rust and general degradation of metal storage and transport materials.

In yet other embodiments, the compositions are formulated for providing one or more of coating onto, infusion into, dispersion within, or formation of articles of manufacture for food service, home goods, and other general use goods, including but not limited to drink dispenser tubing, disposable and reusable drink wear and straws, water, food, and beverage coolers, Denture holders, Mouthguards, sports and Diving/Scuba/swim gear, appliances, and the like.

In accordance with some embodiments, reagents, self-care formulations and kits comprising the compositions may be provided according to the invention. According to some such embodiments, kits comprising one or more individually packaged treatment formulations may be provided, each comprising one or more of treatment implements, such as brushes or other applicators and suspensions comprising the compositions, the treatment formulations provided for application to a surface for applicant to prevent biofilm formation or to treat existing biofilms. And also provided are one or more removal implements, for mechanical removal of biofilms from the surface after application of the treatment formulation. In some examples, the kits are directed to dental care. In other embodiments, the kits are directed to the care of household or consumer products. Accordingly, the kits may further comprise other conventional treatment formulations suited to a particular application.

Compositions

The compositions include, in some embodiments, non-polymerizable antimicrobial mixtures containing a combination of

a) at least one antimicrobially active quaternary ammonium compound, and

b) at least one antimicrobially active quaternary phosphonium compound,

wherein, the combination of components a) and b) are present in a ratio by weight from 1:9 to 9:1.

And wherein the antimicrobially active quaternary ammonium compounds (component a)) are represented by the formula

[R—N*R₁R₂R₃]X⁻ (1)

in which R, R₁, R₂, and R₃ are a preferably straight-chain or branched or cyclic of C2-C20 alkyl radical as same or different length independently; also be as fused cyclic or aromatic ring such as aziridine, azirine, oxaziridine, diazirine, azetidine, azete, diazetidine, pyrrolidine, pyrrole, imidazolidine, imidazole, pyrazolidine, pyrazole, thiazolidine, thiazole, isothioazolidine, isothiazole, piperdine, pyridine, piperzine, diazine, morpholinem oxazine, thiomopholine, thiazine, triazine, triazoles, furanzan, oxadiazole, thiadizole, dithozole, tetrazole, azepane, azepine, diazepine, thiazepine, azocane, azocine, azonane, azonine, etc.

where X⁻ is a—counter anion, which can be inorganic, anions (Cl⁻, AlCl₄⁻, PF₆⁻, BF₄⁻, NTf₂⁻, DCA⁻, etc.) or organic anions (CH₃COO⁻, CH₃SO₃⁻, etc.). These quaternary ammonium compounds can be present in the mixtures according to the invention either individually or in admixture with one another.

And wherein antimicrobially active quaternary phosphonium compounds (component b)) are, in particular, compounds corresponding to the following formula

[RP⁺R₁R₂R₃]Y⁻ (II)

in which R, R₁, R₂, and R₃ are a preferably straight-chain, branched or cyclic of C2-C20 alkyl radical as same or different length independently;

Y⁻ is a halide anion, such as chloride, bromide or iodine anion.

Or according to the formula

[(R′)₃P⁺R″]Y⁻ (III)

in which R′ is a C1-C5 alkyl radical, a C1-C6 hydroxyalkyl radical or a phenyl radical, R″ is a C3-C18 alkyl radical and Y− is a halide anion, more especially a chloride anion or a bromide anion. The radicals R″ and R′″ in formula II are preferably straight-chain or branched or cyclic radicals. The quaternary phosphonium compounds can be present in the mixtures of the invention either individually or in admixture with one another. Examples of quaternary phosphonium compounds of the above type are trimethyl-n-dodecyl phosphonium chloride, triethyl-n-decyl phosphonium bromide, tri-n-propyl-n-tetradecyl phosphonium chloride, trimethylol-n-hexadecyl phosphonium chloride, tri-n-butyl-n-decyl phosphonium chloride, tin-n-butyl-n-dodecyl phosphonium bromide, tri-n-butyl-n-tetradecyl phosphonium chloride, tri-n-butyl-n-hexadecyl phosphonium bromide, tri-n-hexyl-n-decylphosphonium chloride, triphenyl-n-dodecyl phosphonium chloride, triphenyl-n-tetradecyl phosphonium bromide and triphenyl-n-octadecyl phosphonium chloride. Tri-n-butyl-n-tetradecyl phosphonium chloride is preferred.

The compositions also include, in other embodiments, polymerizable antimicrobial mixtures containing at least one type of moieties as defined in I, II, III, the moieties further comprising at least one polymerizable group such as, but not limited to, acrylate, methacrylate, acrylamide, vinyl, vinyl-ether, cyclic ether(epoxy) or cyclic amines and cyclic imine, of which presented as modified R, R₁, R₂, R₃, R′, and R″.

These quaternary ammonium and phosphonium compounds can be present in the mixtures according to the invention either individually or in admixture with one another.

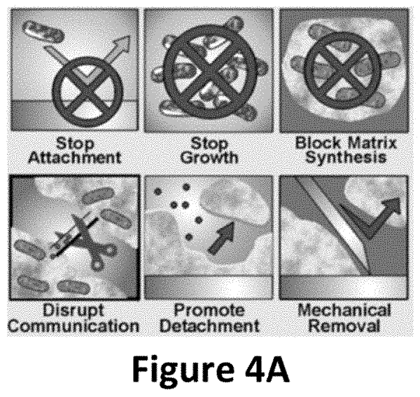

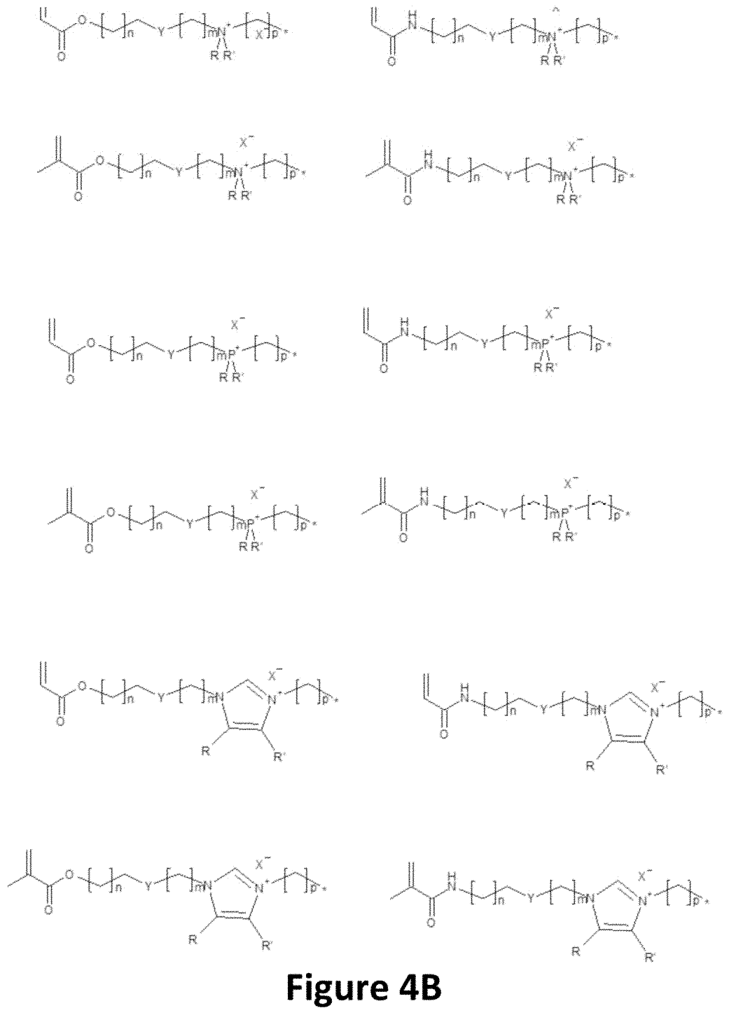

Some specific examples of monomers in accordance with the embodiments hereof are shown in FIG. 4B, wherein:

n, m: same or independently as 0, 1, 2, 3 . . . .

P: 5, 6, 7, 8, 9, 10, 11, 12, 13, 14, 15 . . .

R, R′: same or independently as H, CH₃, C₂H₅CH₂C₆H₅

X: halide, carboxylic acid, sulfonic acid, phosphoric acid, other Lewis acid

Y: direct link, O, S, COO, CONH, CONR, OOCO, OCONH, NHCONH

Monomeric and polymeric resins as disclosed herein may be composed of, in some embodiments, the functional non-polymerizable resins containing at least one of each of antimicrobially active quaternary ammonium and phosphonium compounds, and in other embodiments polymerizable resins containing at least one of antimicrobially active quaternary ammonium and phosphonium compounds at least one polymerizable group, wherein according to the various embodiments, the antimicrobially active quaternary ammonium and phosphonium compounds are present in compositions, articles and coatings in amounts of from about 0.1 weight percent to about 10 weight percent, the amount selected to achieve balanced biofilm attenuating activity, antibacterial activity/microbial cytotoxicity and mechanical properties of the compositions, articles and coatings. Thus, in some embodiments, the antimicrobially active quaternary ammonium and phosphonium compounds are present in amounts from about 0.1 weight percent to about 10 weight percent, and in some embodiments up to 50 weight percent or more, including 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 20.0, 30.0, 40.0 and 50.0 and fractional increments there between.

In accordance with the various embodiments, compositions may be formulated with or incorporated or dispersed in resins known in various arts for forming or coating articles of manufacture. And in accordance with the compositions hereof, resins for composites may be selected from, by way of non-limiting examples, HEMA and HPMA, which are typical monomethacrylate resins; BisGMA, TEGDMA, UDMA are typical conventional dimethacrylate resins, which are polymerizable/curable by heat, light and redox initiation processes. —CQ and LTPO are typical photoinitiators. Tertiary aromatic amines, such as EDAB, may be included as an accelerator for CO-based photoinitiator. Other additives such as inhibitors, UV stabilizers or fluorescent agents may also be used. In addition, a variety of particles, polymeric, inorganic, organic particles may be incorporated to reinforce the mechanical properties, rheological properties, and sometime biological functionalities.

The following abbreviations may be used: BisGMA: 2,2-bis(4-(3-methacryloyloxy-2-hydroxypropoxy)-phenyl)propane HEMA: 2-hydroxyethyl methacrylate HPMA: 2-hydroxypropyl methacrylate TEGDMA: triethylene glycol dimethacrylate UDMA: di(methacryloxyethyptrimethyl-1,6-hexaethylenediurethane BHT: butylhydroxytoluene CQ; cannphorquinone LTPO: lucirin TP0/2,4,6-trimethylbenzoyldiphenylphosphine oxide EDAB: 4-Ethyl dimethylaminobenzonate AMAHP: 3-(acryloyloxy)-2-hydroxypropyl methacrylate EGAMA: ethyleneglycol acrylate methacrylate TCDC: 4,8-bis(hydroxymethyl)-tricyclo[5,2,1,02=6] CDI: 1,1-carbonyl-diimidazole SR295: pentaerythritol tetraacrylate.

### EXPERIMENTAL EXAMPLES

Influence of Composite Material on the Development, 3D Architecture and Mechanical Stability of S. mutans Biofilms

Goal: Examine how the biofilm formation is affected by the test composite in terms of biomass, and how its mechanical stability is changed.

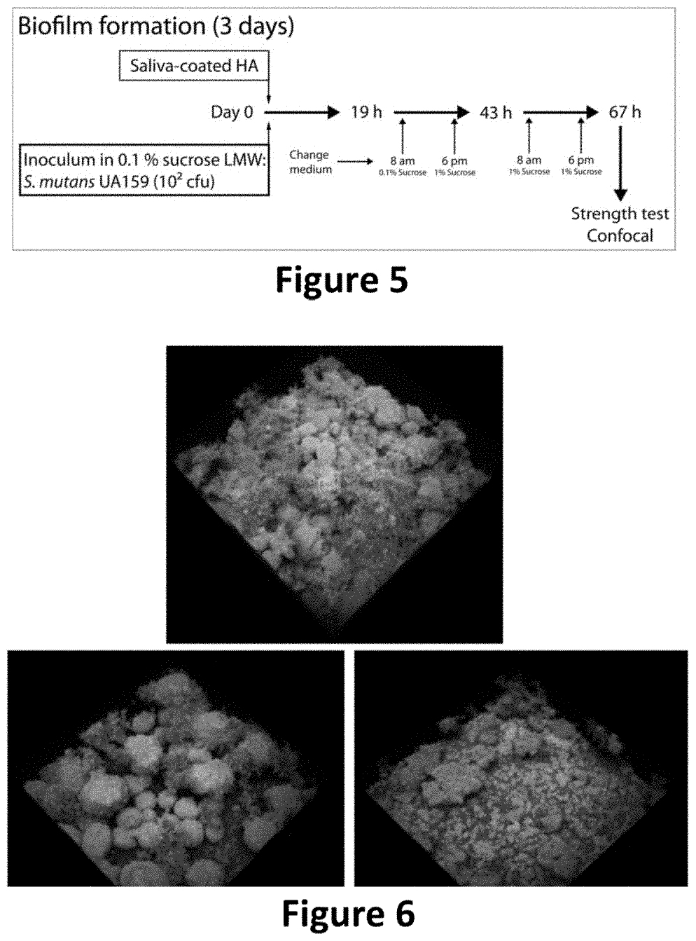

Biofilm growth protocol is shown in FIG. 5.

Test Groups:

HA disc

Conventional Dental Composite/IJ8-095 (control) sterilized by autoclaving

Experimental Antibacterial Composite/IJ8-083 (test) sterilized by autoclaving

Analyses

Inhibition of biofilm formation

Intact biofilm 3D architecture

Intact biofilm biomass (dry-weight)

pH changes of supernatant

Variation of antibiofilm effect

Mechanical stability by applying shear stress

Biofilm removal profile

Sheared biofilm 3D architecture

Analysis of EPS-matrix

EPS-matrix in 2D Cartesian coordinate system (XY, YZ, and XZ planes)

Analysis of EPS-matrix via topological skeleton method

Results

Inhibition of biofilm formation

Intact biofilm 3D architecture

FIG. 6 shows the 3D architecture of 67 h-old biofilms formed on each surface.

Biofilm formation was clearly disrupted by the test composite. Confocal images show that biofilm formation and accumulation were significantly compromised by the test composite.

Use of Saliva Coating Evidenced No Impact on the Antibacterial Effect of the Test Composite.

The composites were sterilized by using 70% EtOH+UV. However, the test composite was much less effective than the autoclaved test composite, and prone to contamination. Therefore, autoclaved composites were used.

Intact Biofilm Biomass

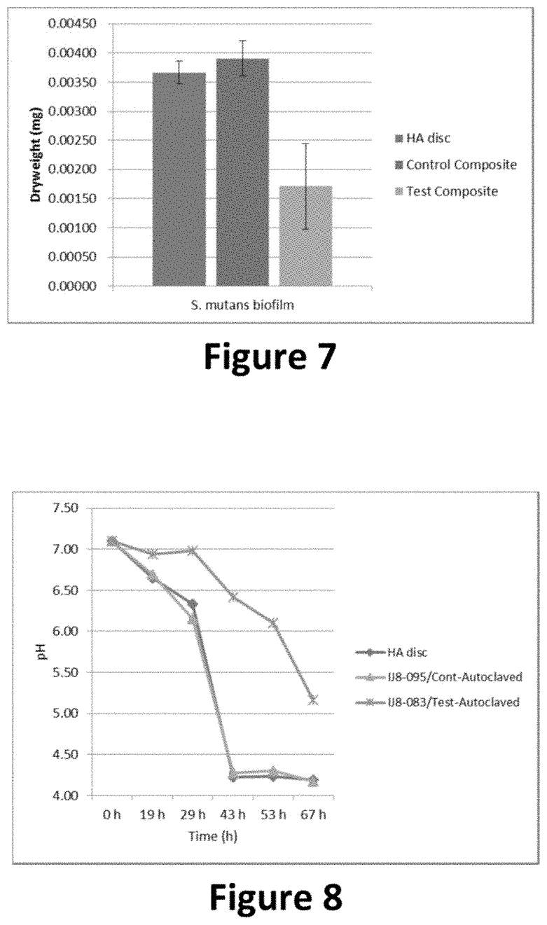

FIG. 7 shows the quantitative data of biomass from each surface.

At 67 h, biomass from the test composite was 2.3 times less than the biomass from control composite, which agrees very well with the confocal imaging data.

Inhibition of biofilm formation was maintained even after the initial biofilm formation period (29 h), indicating lasting effect for prolonged period.

pH Changes

FIG. 8 shows that the pH of the supernatant surrounding test composites was significantly higher than the pH of supernatant of control composite. It indicates that biofilm formation and accumulation were affected during the whole experimental period. However, pH deviation was largely due to some variation of antibiofilm effect.

Variation of the antibiofilm effect can be visualized, and a new finding about potential long-term effect of the material (see later section).

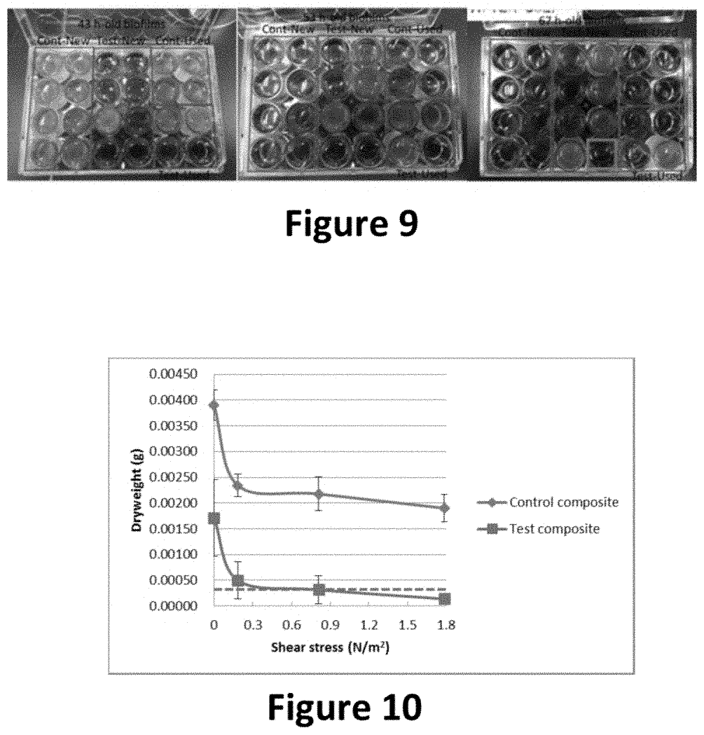

FIG. 9 shows images of the supernatant during biofilm growth

Above images are the 24-well plates containing supernatant during biofilm growth period.

Usually, the supernatant became turbid when the bacterial growth is active in the first 29 to 43 h, then it became clear again (after 53 h) once the biofilm growth became stable.

Between 29-43 h (active bacterial growth transitioning to biofilm phase), all the supernatant from control composite were turbid. Then after 53 h, all the supernatant of control composite became clear, as biofilm growth establishes.

In contrast, all the supernatant from test composite (except one) were mostly clear between 29-43 h, indicating antibacterial activity. However, some variability was observed on the effects after 43 h. indicating variability of the antibacterial release profile among the different test samples.

One supernatant from the test composite (box with green dotted line) never became turbid by the end of biofilm growth (67 h), indicating strong antibacterial activity and no biofilm growth on the surface.

Additional Information:

Further analyses were conducted to determine if the used test composite would be effective. Surprisingly, re-used test composites were still interfering with the initial biofilm formation and accumulation, which suggests a long term effect even after re-use.

Mechanical Stability

Biofilm Removal Profile

FIG. 10 shows the remaining biomass from each composite surface after applying shear stress (n>=12).

Biomass removal patterns were similar, while the amount of biomass from the test composite was significantly lower than the one from the control composite.

At 0.804 N/m², biofilm removal from the test composite already reached a detection limit (˜0.0003 g), while the percentage of biomass removal from the control composite was still only ˜50%. There was no significant further removal from the control composite at 1.785 N/m².

Sheared 3-D biofilm architecture

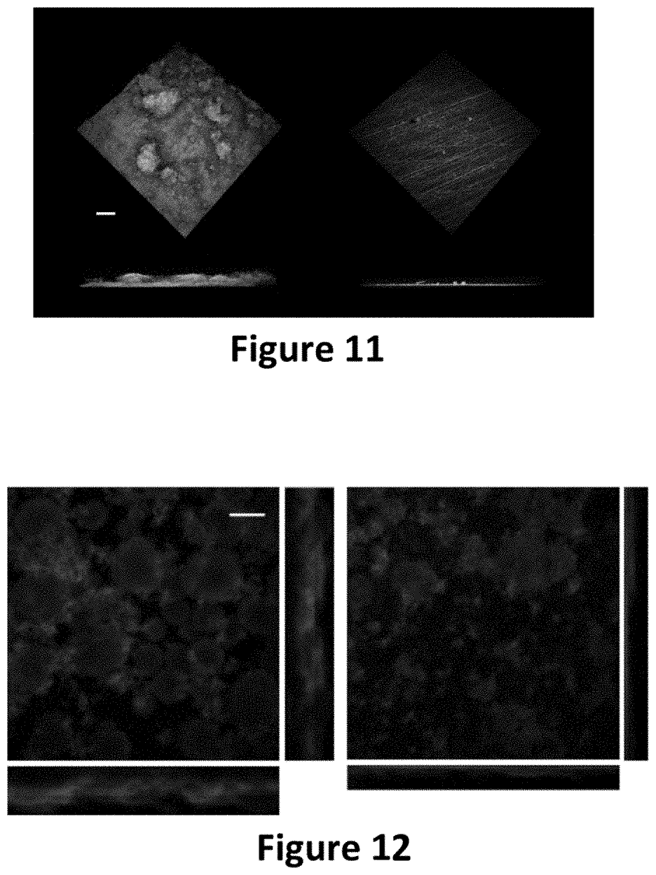

FIG. 11 shows the representative confocal image of 67 h biofilms after exposure to shear stress of 0.804 N/m².

Although biofilms on the control composite were flattened under application of shear force of 0.804 N/m², numerous bacterial microcolonies still attached to the control composite.

Strikingly, most of the bacterial biomass and EPS-matrix on the test composite were clearly removed, while a few tiny aggregates remained.

Quite Surprisingly, the Results Show that Dental Composites Comprising the Compositions According to the Invention can Disrupt Both the Initial Biofilm Formation and its Further Development. Although Biofilms are not Completely Inhibited on the Test Composite, the biofilm Accumulated can be Easily Removed and Detached by Low External Shear Forces.

Analysis of EPS-Matrix

FIG. 12 shows EPS-matrix in 2-D Cartesian coordinate system (XY, YZ, and XZ planes)

To understand why the biofilms on the test composite are easily removed, the structural morphology of EPS-matrix was assessed. FIG. 12 shows the representative projection images of intact 67-h biofilms in XY, YZ, and XZ planes.

EPS-matrix on the control composite was thick and relatively evenly distributed over the entire surface. Also, the EPS-matrix is structurally more organized, which appeared to be connected to each other forming a network that likely provides a strong and stable architecture.

In contrast, the EPS-matrix on the test composite was much thinner compared to the matrix on the control composite. Further, the shape of the matrix appeared scattered and unorganized. It may indicate lack of structural stability (in sharp contrast to control composite) of the scattered EPS-matrix formed on the test composite.

Additional analyses were conducted to verify whether there was significant differences in the geometrical pattern of the EPS formed on control vs test composite surfaces.

Analysis of EPS-Matrix Via Mathematical Morphology

To further analyze the structure of EPS-matrix, the topological skeleton method was applied which is based on theoretical analysis and processing of geometrical structures. The skeleton usually emphasizes geometrical and topological properties of the shape, such as its connectivity, topology, length, direction, and width. Thus, it can provide basic information regarding how the EPS-matrix is developed and organized.

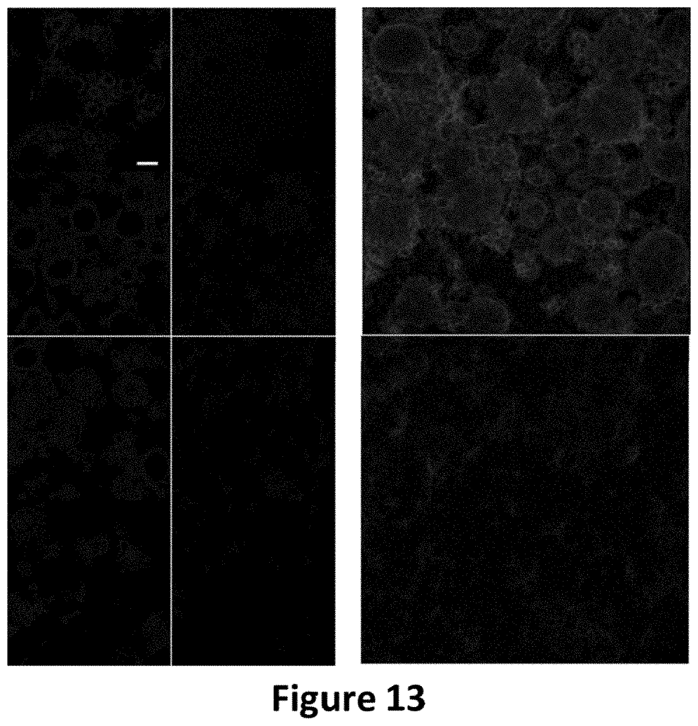

FIG. 13 demonstrates that the projected image of skeletonized EPS-matrix on the control composite is clearly a well-structured surrounding EPS-matrix that is connected by thick filaments, while the inside structure is densely filled with thin filaments. Clearly, the assembly of the entire EPS-matrix is highly organized, which may explain the mechanical resistance of biofilm to external shear forces.

In contrast to the control composite, the EPS-matrix on the test composite was devoid of thick filaments, but rather thin and short filaments without any pattern were observed. At 40 μm height, the EPS-matrix was already disconnected and its density was reducing with increased height. The projection image shows poorly developed overall EPS-matrix which may not be able to withstand external shear forces.

Collectively, the Test Composite May Impede the Formation of a Typical EPS-Matrix with Densely Packed Thick and Thin Filaments that Provides Strong Resistance to Mechanical Stress.

As used herein, the singular forms “a,” “an,” and “the” are intended to include the plural forms as well, unless the context clearly indicates otherwise.

As used herein the term “biofilm” refers to an extracellular polymeric substance produced by and including microbes and having three-dimensional structural characteristics. Biofilms, whether on a surface or in a suspension, provide a matrix that can support the retention and growth of one or more of discrete microbial species and mixed species populations selected from bacteria, fungi, protozoa, algae, and others. In some embodiments, biofilms comprise co-aggregating organisms.

The term “coating” as used herein refers to a topically applied or superficial layer or surface of an underlying material that constitutes a material covering an article such as a medical device, a dental composite or apparatus, a container such as for food or industrial goods, and the like.

As used herein, the term “microbe” refers to a microorganism and is intended to encompass both an individual organism, and hetero and homogenous populations comprising any number of the organisms. As used herein, the term “microorganism” refers to any of a variety of species or microorganism, including but not limited to, archaea, bacteria, fungi, protozoans, mycoplasma, and parasitic organisms, wherein the term “fungi” is used in reference to eukaryotic organisms such as the molds and yeasts, including dimorphic fungi, and the terms “bacteria” and “bacterium” refers to the various examples as specifically disclosed in the tables and description herein, broadly including prokaryotic organisms within the phyla in the kingdom Procaryotae, the microorganisms including Actinomyces, Chlamydia, Streptomyce, and all cocci, bacilli, spirochetes, spheroplasts, protoplasts, all Gram-negative and Gram-positive “Gram-negative” and “Gram-positive” refer to staining patterns with the Gram-staining process, and all non-pathogenic bacteria and pathogenic bacteria. In particular, the term “pathogen” refers to a biological organism that causes or to which can be at least partially attributed any of a variety of disease states in a host, and include, but are not limited to, archaea, bacteria, fungi, protozoans, mycoplasma, parasites, and viruses.

As used herein, the term “antimicrobial agent” refers to composition that decreases, prevents or inhibits the growth of bacterial and/or fungal organisms. In some specific examples of antimicrobial agents, antibiotics are those substances that inhibit the growth of microorganisms, ideally without damage to the host. In various different examples, antibiotics may affect one or more of a microbial cell's activity resulting in cell death, including but not limited to inhibition or alteration of one or more of membrane function and nucleic acid, protein, and cellular component/cell wall synthesis. Antibiotics can include, but are not limited to, macrolides (e.g., erythromycin), penicillins (e.g., nafcillin), cephalosporins (e.g., cefazolin), carbapenems (e.g., imipenem), monobactam (e.g., aztreonam), other beta-lactam antibiotics, beta-lactam inhibitors (e.g., sulbactam), oxalines (e.g., linezolid), aminoglycosides (e.g., gentamicin), chloramphenicol, 15 sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, rifamycins (e.g., rifampin), streptogramins (e.g., quinupristin and dalfopristin) lipoprotein (e.g., daptomycin), polyenes (e.g., amphotericin B), azoles (e.g., fluconazole), and echinocandins (e.g., caspofungin acetate). Examples of specific antibiotics include, but are not limited to, amifloxacin, amphotericin B, and nystatin, azithromycin, aztreonam, cefazolin, ciprofloxacin, clarithromycin, clavulanic acid, clinafloxacin, clindamycin, enoxacin, erythromycin, fleroxacin, fluconazole, gatifloxacin, gemifloxacin, gentamicin, imipenem, itraconazole, ketoconazole, linezolid, lomefloxacin, metronidazole, minocycline, moxifloxacin, mupirocin, nafcillin, nalidixic acid, norfloxacin, ofloxacin, pefloxacin, rifampin, sparfloxacin, sulbactam, sulfamethoxazole, teicoplanin, temafloxacin, tosufloxacin, trimethoprim, vancomycin.

As used herein, the term “medical devices” includes any material or device that is used on, in, or through a subject's or patient's body, for example, in the course of medical treatment to address, to minimize or prevent an illness or injury. Medical devices include, but are not limited to, such items as CPAP, Ventilation equipment, Central lines, Kwires and screws for fracture fixation, and orthopedic reduction or distraction and other medical implants, catheters, intravascular catheters, dialysis shunts, wound drainage tubes, skin sutures, vascular grafts, implantable meshes, intraocular devices, heart valves, graft materials, needles, transdermal and transmucosal patches, sponges, and personal care and hygiene products selected from but not limited to tampons, sponges, intrauterine devices, diaphragms, condoms, gloves, drapes and films, wound dressings, tapes and dressings, and the like.

Dental devices include, but are not limited to Dental Composite, Dental Adhesive, Dental Cement, Dental Sealant, Dental Liner, Dental Varnish, Denture. Root Canal Sealer, Implant Cement, Orthodontic Cement, Self-disinfected Dental Impression Material, Wearable or removable dental plaque treatment device (Antibacterial Night Guard). According to such embodiments, the compositions can be used in Resin Composite-based CAD/CAM Blocks; for Temporary Crown-bridge Composite: for—Pediatric Crown: for Esthetic Orthodontic Aligner: for Esthetic Polymer based Orthodontic Bracket (and maybe coating for metal/ceramic bracket); and in some particular embodiments, the compositions can be used in Coating for Dental Implant Abutment. And according to other such embodiments, the compositions may be provided in suspension or coated on micro or nanoparticles for use in mouthwashes, dental strips, dental films and gels, toothpaste and other dental care items.

The general inventive concepts herein are described with occasional reference to the exemplary embodiments of the invention. Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts.

Unless otherwise indicated, all numbers expressing quantities, properties, and so forth as used in the specification and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts or features into additional embodiments and uses within the scope of the general inventive concepts even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure: however, such values and ranges are not to be construed in a limiting sense and are intended to be critical values or ranges only if so expressly stated.

What is claimed is:

1. A composition for attenuating biofilms, comprising:
a mixture that includes one or more polymerizable quaternary ammonium and one or more polymerizable quaternary phosphonium compounds, present in a ratio of polymerizable quaternary ammonium compounds to polymerizable quaternary phosphonium compound, by weight, in a range from 1:9 to 9:1,
wherein the one or more polymerizable quaternary ammonium compound is represented by the formulas (i)-(vii): 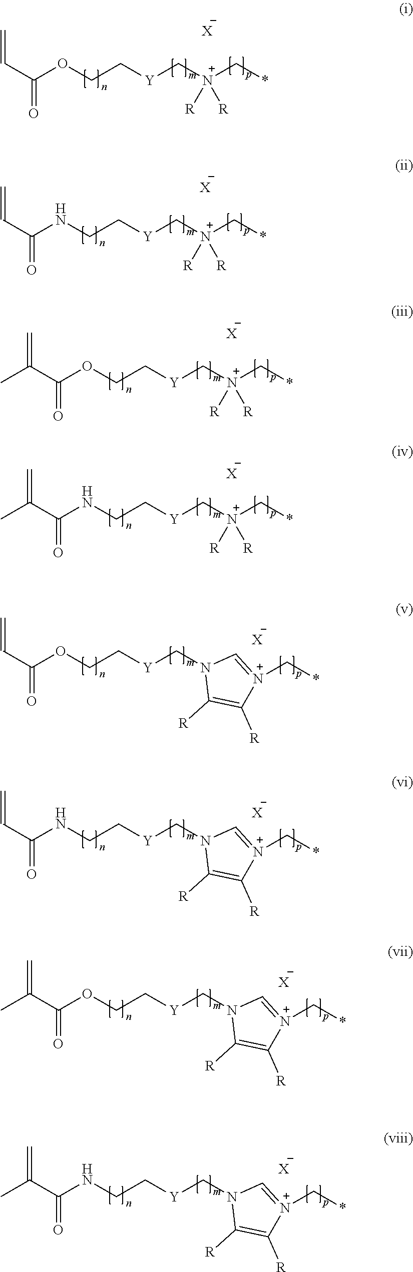
wherein n=0, 1, 2, or 3; m=0, 1, 2, or 3; p=5, 6, 7, 8, 9, 10, 11, 12, 13, 14, or 15; R, R′ same or independently=H, CH₃, or C₂H₅CH₂C₆H₅; X=a halide, or one of a carboxylic, sulfonic, phosphoric, or Lewis acid; and Y=direct link, O, S, COO, CONH, CONR, OOCO, OCONH, or NHCONH; and
wherein the one or more polymerizable quaternary phosphonium compound is represented by the formulas (ix)-(xiii): 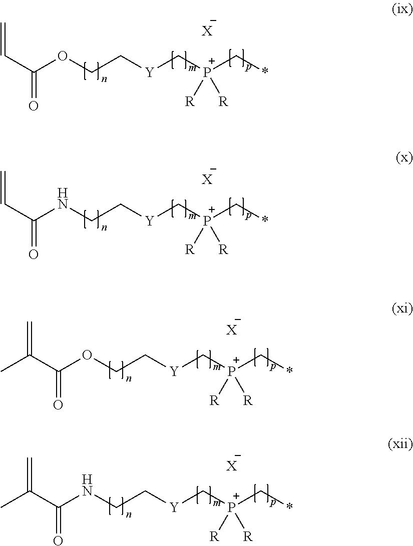
wherein n=0, 1, 2, or 3: m=0, 1, 2, or 3; p=5, 6, 7, 8, 9, 10, 11, 12, 13, 14, or 15; R, R′ same or independently=H, CH₃, or C₂H₅CH₂C₆H₅; X=a halide, or one of a carboxylic, sulfonic, phosphoric, or Lewis acid; and Y=direct link, O, S, COO, CONH, CONR, OOCO, OCONH, or NHCONH; and
[RP⁺R₁R₂R₃]Y⁻ (xiii)
wherein R is a cyclic radical comprising vinyl as a polymerizable, R₁ and R₂ are straight chain or branched C2-C6 alkyl radicals, R₃ is a C8-C12 straight chain alkyl radical, and Y⁻ is an inorganic or an organic anion, and
wherein the composition for attenuating biofilms is capable of disruption of an initial formation of biofilm and further development of the biofilm such that the accumulated biofilm can be removed by a low shear force of less than 1.785 N/nm².

2. The composition for attenuating biofilms according to claim 1, wherein the one or more polymerizable quaternary ammonium compound includes: 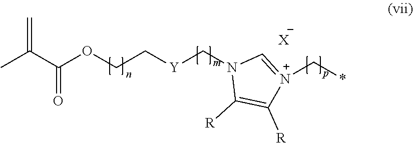
wherein n=0, 1, 2, or 3; m=0, 1, 2, or 3; p=5, 6, 7, 8, 9, 10, 11, 12, 13, 14, or 15; R, R′ same or independently=H, CH₃, or C₂H₅CH₂C₆H₅; X=a halide, or one of a carboxylic, sulfonic, phosphoric, or Lewis acid; and Y=direct link, O, S, COO, CONH, CONR, OOCO, OCONH, or NHCONH.

3. The composition for attenuating biofilms according to claim 1, wherein Y in the one or more polymerizable quaternary phosphonium compound is a chloride, bromide, or iodine anion.

4. The composition for attenuating biofilms according to claim 1, wherein each of the one or more polymerizable quaternary ammonium compound and the one or more polymerizable quaternary phosphonium compound is present in the composition in an amount that in a range from about 0.1 to 10% by weight based on the total weight of the composition.

5. The composition for attenuating biofilms according to claim 1, wherein the one or more polymerizable quaternary ammonium compound and the one or more polymerizable quaternary phosphonium compound is each present in the composition up to 50% by weight based on the total weight of the composition.

6. The composition for attenuating biofilms according to claim 1, wherein the one or more polymerizable quaternary ammonium compound is present in the composition at 50% by weight based on the total weight of the composition and the one or more polymerizable quaternary phosphonium compound is present in the composition at 50% by weight based on the total weight of the composition.

7. An article of manufacture comprising the composition according to claim 1, wherein the composition is polymerized and formed as a solid article, is dispersed within a solid article, or is formed as a coating on a solid article, the solid article selected from a dental implant, a dental device, a medical device, and a personal care device.

8. A resin blend comprising the composition according to claim 1 dispersed with one or more resins.

9. An article of manufacture comprising the resin blend according to claim 8, wherein the article of manufacture is selected from dental composite, dental adhesive, dental cement, dental sealant, dental liner, dental varnish, denture, root canal sealer, implant cement, orthodontic cement, self-disinfected dental impression material, a wearable dental plaque treatment device, and a removable dental plaque treatment device.

10. An article of manufacture, comprising:
(a) one or more individually packaged treatment implements, such as brushes or other applicators, (b) one or more individually packaged compounds of the composition according to claim 17, and (c) one or more removal implements for mechanical removal of biofilms from the surface after application of the treatment formulation.

11. A composition for attenuating biofilms, comprising:
a mixture that includes one or more polymerizable quaternary ammonium and one or more polymerizable quaternary phosphonium compounds, present in a ratio of polymerizable quaternary ammonium compounds to polymerizable quaternary phosphonium compound, by weight, in a range from 1:9 to 9:1,
wherein:
the one or more polymerizable quaternary ammonium compound includes 1-[2′-(methacryloyloxyl)-ethyl propanoate]-3-dodecylimidazolium bromide;
the one or more polymerizable quaternary phosphonium compound includes (4-vinylbenzyl)-dibutyl dodecyl phosphonium chloride; and
the composition for attenuating biofilms is capable of disruption of an initial formation of biofilm and further development of the biofilm such that the accumulated biofilm can be removed by a low shear force of less than 1.785 N/m².
